(19) United States
(12) Patent Application Publication
(10) Pub. No.: US 2024/0024113 A1
(43) Pub. Date: Jan. 25, 2024

(54) USE OF LIQUID CRYSTAL ELASTOMERS IN ORTHOPEDIC APPLICATIONS

(71) Applicant: Impressio Inc., Denver, CO (US)

(72) Inventors: Amir H. Torbati, Westminster, CO (US); Lyssa A. Bell, Littleton, CO (US); Lillian S. Chatham, Lakewood, CO (US); Josh Ray, Dallas, TX (US); Rajib K. Shaha, Hillsboro, OR (US); Aura Penalosa, Denver, CO (US); Ross H. Volpe, San Diego, CA (US); Richard M. Wojcik, Denver, CO (US); Christopher M. Yakacki, Denver, CO (US); Risheng Zhou, Denver, CO (US)

(21) Appl. No.: 18/206,821

(22) Filed: Jun. 7, 2023

Related U.S. Application Data
(60) Provisional application No. 63/349,902, filed on Jun. 7, 2022.

(51) Int. Cl. A61F 2/30

(52) U.S. Cl. CPC A61F 2/30756; A61F 2002/4233

(57) ABSTRACT

A device for a metatarsophalangeal (MTP) joint includes a body with a stem and a head. The stem may be hollow and include a plurality of perforations about its length to improve osseointegration. The head may include one or more perforations and/or one or more islands. The one or more perforations and one or more islands are configured to promote bonding the body and liquid-crystalline elastomer (LCE) that is applied to the head. The device may be inserted into bone to form an MTP joint replacement.

### CROSS-REFERENCE TO RELATED APPLICATIONS

This patent application claims priority from, and incorporates by reference the entire disclosure of, U.S. Provisional Patent Application No. 63/349,902 filed on Jun. 7, 2022.

### STATEMENT REGARDING FEDERALLY SPONSORED RESEARCH

This invention was made with government support under awards 2014661, 1827288, 1654807, and 1350436 by the National Science Foundation. The government has certain rights in the invention.

### TECHNICAL FIELD

The present disclosure relates generally to the use of liquid-crystalline elastomer (LCE) and more particularly, but not by way of limitation, to the use of LCEs to mimic biological tissues.

### BACKGROUND

This section provides background information to facilitate a better understanding of the various aspects of the disclosure. It should be understood that the statements in this section of this document are to be read in this light, and not as admissions of prior art.

Approximately 2.5% (1 in 40) of people over 50 years old develop osteoarthritis of the first metatarsophalangeal (“MTP”) joint, called hallux rigidus. Thus, around 2-3 million people have hallux rigidus in the U.S. In 2014 alone, over 20,000 people in the U.S. were diagnosed with hallux rigidus. Hallux rigidus is the most common arthritic condition in the foot. The exact cause of hallux rigidus has not been determined, but it may result from several conditions, such as neuromuscular disorders, metabolic disorders, congenital disorders, acute trauma, or repetitive trauma to the articular cartilage of the MTP joint. Other studies have shown that hallux rigidus usually presents in an idiopathic manner. Regardless of the underlying disease process, there are degenerative changes in the joint, including loss of articular cartilage, narrowing of the joint space, and osteophyte formation.

A number of techniques, including arthrodesis, arthroplasty, and hemi-arthroplasty are aimed at treating end-stage hallux rigidus; however, each technique has its own drawbacks. Recently, a synthetic cartilage device based on a poly(vinyl alcohol) hydrogel was FDA approved for treatment of hallux rigidus as a joint-salvaging option. However, even this device has had numerous clinical failures (˜30%), such as device tears and fractures, synovial reactions, device related bone erosion, and subsidence into the metatarsal bone causing bone-on-bone contact.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

The unique properties of LCE material provide the ability to mimic the structure and properties of cartilage by (1) dissipating large amounts of energy, (2) controlling anisotropy within the material, and (3) having tunable mechanical properties by changing the crosslinking density to match the modulus of soft tissues. LCEs are unique in their ability to dissipate energy over a large frequency range, rather than the limited dissipation observed in most elastomers (i.e., silicones and hydrogels). The LCE are tailored with liquid crystal ordered structures and crosslinking density to closely mimic the mechanical properties of cartilage. The LCEs can be utilized to replace MTP joints to better replicate the native function by exploiting LCE's inherent soft, dissipative, and anisotropic behavior. The technological innovation is derived from the design of an LCE, capable of minimal wear while sustaining the forces experienced in the joint.

LCEs have vastly superior energy dissipation properties relative to traditional elastomers such as silicone or hydrogels. LCEs can be used to treat degenerated joints by combining the fields of liquid-crystal physics, polymer viscoelasticity, and bioengineering. Main-chain LCEs are defined by synthesizing liquid-crystal mesogens directly into the polymer backbone; mesogen reorientation under mechanical stress provides a temperature and frequency insensitive mechanism in the body at body temperature and a low frequency for walking/running for dissipating energy that is more robust than typical viscoelasticity. LCEs are known for their unique behavior that is similar to biological tissues, however very few LCE applications have been developed in the past due to their notoriously difficult synthesis process. We have recently developed a facile two-stage reaction to synthesize LCEs at scale.

The unique nature of LCEs permits devices to mimic the naturally soft tissues of the body, and provide anatomically correct support. In addition to the ability to mimic the properties of the MTP joint, other potential advantages of using an LCE-based MTP that leads to broader impacts include: (1) Minimally Invasive Surgery—LCEs can undergo strains of over 100% allowing for minimally invasive implantation as they could be inserted via catheter and expanded within the joint space rather than removing the entire joint, (2) Patient specific Devices—The inherent tailorability of LCE mechanical properties enables patient specific device design to provide support in specific areas depending on the patient, (3) Other applications of LCEs—The technology's broader impact stems from its potential to treat arthritis in other joints in the foot, hand, knee (e.g. total knee replacement), spine (e.g. total disc replacement), osteochondral defect (OCD), spherical interpositional arthroplasty, and repair of any load bearing orthopedic tissue such as meniscus.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A and FIG. 1B illustrates mesogen orientation in polydomain (left—FIG. 1A) and monodomain (right—FIG. 1B) main-chain LCEs;

FIG. 2 illustrates a two-stage thiol-acrylate reaction to synthesize and program LCE: (a) illustrates chemicals used for synthesis, (b) illustrates a polydomain state after the Michael addition reaction, (c) illustrates stretching used to orient the mesogens into a monodomain, and (d) illustrates an anisotropic monodomain after UV-crosslinking;

FIG. 3 shows a diagram of collagen arrangement of articulate cartilage (a), and a diagram depicting alignment of mesogens in an LCE-based MTP joint mimicking the structure of an intact cartilage (b);

FIGS. 4A-4C illustrate a process for 3D Printing LCEs using the two-step reaction to make polydomain and monodomain prototypes for patient specific orthopedic implants;

FIG. 5A illustrates an MTP device and FIG. 5B illustrates the MTP device with an LCE cap according to aspects of the disclosure;

FIG. 6A illustrates an MTP device with an LCE cap and FIGS. 6B-6D illustrate the MTP device with the LCE cap removed; and

FIGS. 7A-7F illustrate variations of the design of the MTP device of FIG. 5A.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Reference will now be made to more specific embodiments of the present disclosure and data that provides support for such embodiments. However, it should be noted that the disclosure below is for illustrative purposes only and is not intended to limit the scope of the claimed subject matter in any way.

One of the main problems with existing MTP devices is the mismatch in mechanical properties between the device and an actual joint or the surfaces in contact with the device which leads to failure. Additionally, high stiffness, lack of energy dissipation/cushioning, and lack of mechanical anisotropy leads to uneven stress distribution and increases the risk of subsidence, loosening, and eventually failure. An LCE-based cartilage replacement device for the MTP joint to treat hallux rigidus is unique in its ability to dissipate energy over a large frequency range, rather than the limited dissipation observed in most elastomers (i.e., silicones and hydrogels). The v is tailored with ordered structures to closely mimic the structure of cartilage. LCEs can be utilized to replace MTP joints that better replicate the native function by exploiting their inherent dissipative and anisotropic behavior. The technological innovation is derived from the design of an LCE, capable of minimal wear while sustaining the forces experienced in the joint. The technology's broader impact stems from its potential to treat arthritis in other joints in the foot, hand, and knee. In wear testing of pin (LCE) on disc (CoCr), the LCE did not show any mass loss after it was tested for 2 million cycles at 1 Hz at 37° C. in Bovine Serum at ˜3 MPa stress. The samples were pre-soaked in bovine serum prior to the test and they only gain 3 wt % mass. This mass gain during 2 million cycle testing is in addition to pre-soaked. Weight change versus the number of cycles for UHMWPE was also tested. The control sample was UHMWPE pin on CoCr disc. The flat UHMWPE lost weight, while the round UHMWPE gained weight.

The overarching challenge in treating hallux rigidus involves collectively addressing two current issues: reducing pain and restoring mobility for the patients. Patients with end stage hallux rigidus can be in tremendous pain (average visual analogue scale pain score 70/100), thus decreasing their pain is critical. While dorsiflexion range of motion is not restored in arthrodesis procedures, the current arthroplasty procedures have only a moderate improvement in dorsiflexion angle (range 26-30° post-op). Thus, restoring joint mobility to the full range of motion is critical for active patients to regain function in their daily activities. A goal of the LCE-based cartilage replacement device is to create a novel device that minimally wears and mimics the structure of native cartilage with its anisotropic structure.

Joint replacement technology is limited by current materials. These materials don't mimic the natural motion, cushioning, and shock absorbing properties provided by soft tissues. As a result, implants alter the mechanics of the joint and are susceptible to limited wear life and failure. LCE material has the unprecedented ability to act as both a viscous liquid and rubber simultaneously. Combined with the ability to tailor its structure, LCE materials possess many of the same properties of biological tissues and offers the following advantages discussed below.

Shock Absorption: Current joint replacements can fail when exposed to impacts. This is due to the use of hard brittle materials or hydrogels. LCE materials offer the ability to provide cushioning and shock absorption, which can help avoid implant failure.

Increased Wear Life of Joint Replacement: Extending the life of joint replacements has been a highly sought after “Holy Grail”. However, the mechanics of the joint cannot be replicated using conventional materials such as rubbers and hydrogels. LCE materials can allow micro-motion and cushioning to lengthen the lifetime of the implant and reduce wear. This mainly happens due to having an oval shaped rigid liquid crystal mesogen that can rotate and dissipate energy and heat internally without transferring it to the adjacent environment.

A Broad Platform for Soft Tissues and Joints: The LCE-based cartilage replacement devices have direct follow-on potential in the knee and spinal disc as well as foot and ankle.

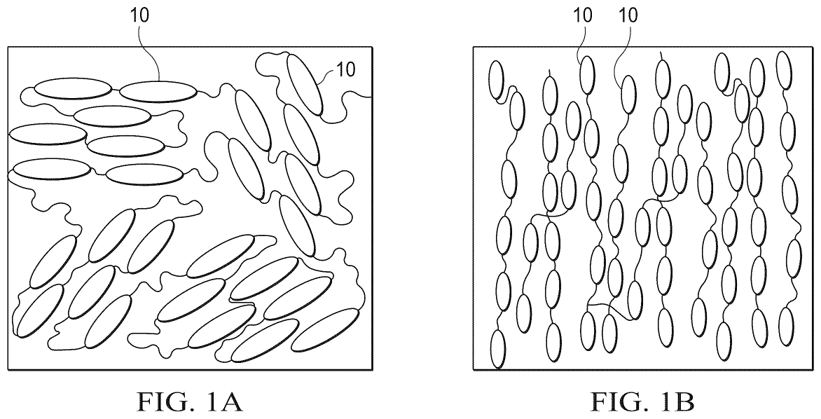

LCEs are a class of multi-functional polymers that combine network elasticity with liquid-crystalline order. Liquid crystals are comprised of rigid aromatic rings known as mesogens. Liquid-crystal order arises when mesogens self-organize, which can exist in two states: polydomain and monodomain. FIG. 1A illustrates the polydomain state in which mesogens 10 are arranged into randomly oriented domains that provide significant damping and energy dissipation. FIG. 1B illustrates the monodomain state in which the mesogens 10 are aligned to increase strength, while being mechanically anisotropic. When locked into a polymer network, mesogen order gives the material unique abilities such as tailored gradients and anisotropy, reversible actuation like a muscle, and the ability for extreme energy dissipation compared to traditional hydrogels, rubbers and elastomers.

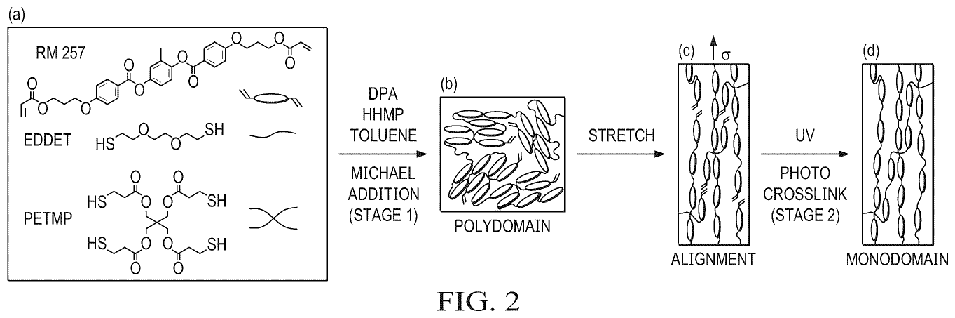

LCE synthesis and preparation is not a trivial process. Main-chain LCEs are defined by synthesizing the mesogens directly into the polymer backbone and show ideal coupling behavior leading to extraordinary properties; however, this process has been a longstanding challenge in the field of LCEs. The majority of researchers use a step-growth hydrosilylation reaction, which requires high-purity materials and careful experimental conditions. To tailor the liquid-crystalline structure into a monodomain, mesogens must be manually oriented during synthesis. Using hydrosilylation reactions, one technique is to allow the material to gel, stretch the gel to orient the mesogens and chains, and then allow the reaction to finish crosslinking the material. This technique is inherently difficult to repeat. Non-mechanical methods such as surface alignment and magnetic fields can also be applied to align mesogens into a monodomain. These methods must be used for one-step reactions that cannot be stopped and restarted, such as free radical reactions. Also, these reactions are limited to samples less than 100 μm thick. Recently, we have developed a new technique to create tailored main-chain LCEs with unprecedented control over scalability, thermo-mechanical properties, and mesogen order. FIG. 2 illustrates a two-stage thiol-acrylate reaction with which large LCE samples can be synthesized and tailored to exploit the unique properties of LCEs (a-d).

LCEs offer an innovative solution to MTP joint replacement by taking advantage of its unique material properties, which are inherently similar to biological materials. Specifically, LCEs are hierarchical in structure, consisting of both mesogen and network length scales; they can be either mechanically isotropic or anisotropic depending on their order and structure; and they have high levels of energy dissipation and damping; and can exist with a gradient in properties. Traditional materials, such as silicone or hydrogels, simply cannot match the properties of natural tissues or LCEs.

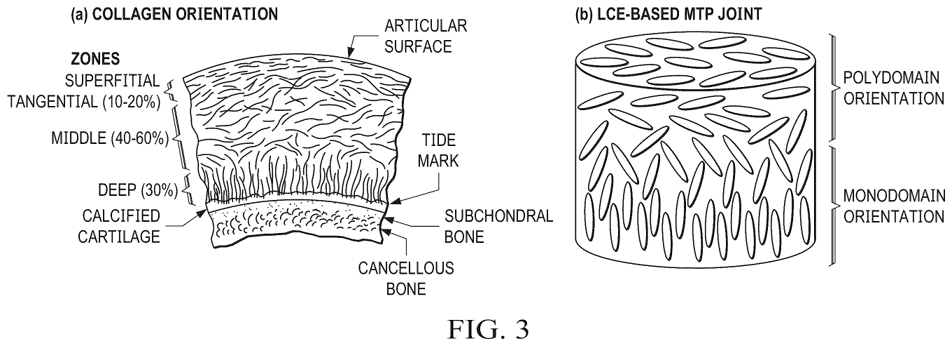

A depiction of how an LCE could be designed to mimic the MTP joint is shown in FIG. 3. The surface of the LCE joint is designed in a manner that leaves the mesogens and network randomly oriented (polydomain orientation) and as a result provides mechanical damping and shock absorption similar to the natural joint. The inner portion of the LCE joint is made of oriented mesogens (monodomain orientation), which will simulate the oriented fibers of the intact MTP joint and provide enhanced longitudinal compressive strength.

Orienting liquid crystal mesogens into a polydomain (randomly oriented mesogens) and monodomain (highly aligned mesogens), function to MTP joints can be restored. Furthermore, LCE-based joint replacement increases durability and reduces wear as compared to the current joint replacements. This approach deviates substantially from currently used conventional materials in MTP devices. This solution will be attractive to surgeons as it does not change the surgical procedure of implantation or overall design.

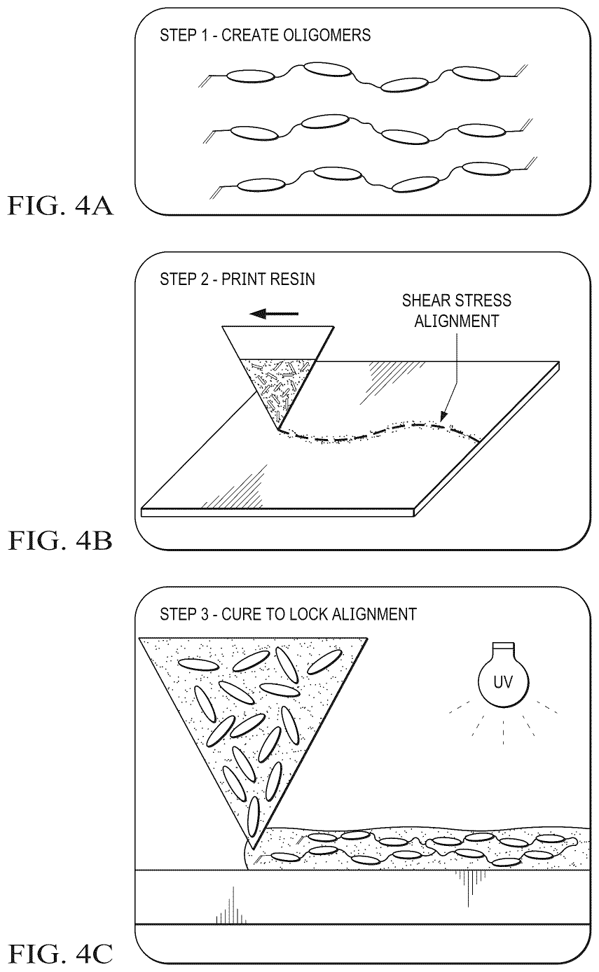

In addition to the ability to mimic the structure of the natural joint, other potential advantages of using an LCE-based MTP joint include: Patient Specific Devices—The inherent tailorability of LCE mechanical properties enables patient specific device design via 3D printing to support specific areas depending on patient radiographic images. FIGS. 4A-4C illustrate a 3D-printing method. The method includes creating oligomers (FIG. 4A), printing resin comprising the oligomer on a substrate (FIG. 4B), and curing the printed resin/oligomer mixture via application of UV light (FIG. 4C). 3D printing will be utilized to make patient specific molds, and 3D printing can be used to print patient specific LCEs. Using implants with a porous surface would permit additional areas for bone ingrowth, securing the implant over time; again, reducing the likelihood of expulsion.

Reference will now be made to particular materials and methods utilized by various embodiments of the present disclosure. However, it should be noted that the materials and methods presented below is for illustrative purposes only and is not intended to limit the scope of the claimed subject matter in any way.

Working Example 1

Task 1: Synthesize Tailored LCEs

A library of LCE polymers will be synthesized by varying the amount of liquid crystals and crosslinking in the polymer chains for both static (modulus and ultimate strength) and dynamic (phase lag) loading, as well as dynamic behavior of LCEs under cyclic loading. Completion of this task will offer the first material with the ability to match the shock absorption and wear performance of cartilage in the natural MTP joint.

Using the two-stage reaction developed by our team, we can control mechanical properties by changing the crosslinking density during synthesis. Furthermore, using a second stage photo-crosslink reaction, the hierarchical arrangement of the liquid-crystalline domain can be tailored to achieve a material that can closely mimic the MTP joint with respect to anisotropy, alignment, and modulus. This will be the first study to investigate the use of LCEs for a load-bearing joint replacement. The FDA recommends cyclic testing for 5 million and 10 million cycles as the worst-case scenario for joint replacements. Therefore, the long-term mechanical performance of these materials will be investigated.

Experimental Design

Traditionally, the manufacturing of these systems has been a major challenge in the application of these materials. Using the chemistry shown in FIG. 2, the initial modulus of the materials is tailored by varying the crosslink density using 35, 45, 55, and 65 mol % PETMP crosslinker. The synthesis method is scalable and can be cured in molds that closely mimic the size of MTP joints.

Anisotropy and further mechanical tailoring will be achieved by using a second-stage photo-polymerization reaction. By utilizing an excess of acrylate groups during polymerization, samples can be mechanically manipulated and locked into shape using UV light. Cylindrical polydomain samples will be mechanically stretched to 50, 70 and 90% strain to create monodomain samples with different degree of anisotropy. During stretching, the outer portion of the LCE samples will experience a high degree of circumferential strain and alignment of mesogens, due to conservation of volume (v=0.5) (as shown in FIG. 2). Once stretched, 365 nm UV light with an intensity between 10-20 mW/cm² will be used to photo-crosslink the samples and lock in the desired anisotropy.

After all samples have been synthesized, they will undergo (i) quasi-static mechanical compression to measure compressive modulus and failure stress (n=5/group) and (ii) dynamic (cyclic) mechanical compression to measure storage modulus, phase lag δ, and cycles to failure (n=5/group). In general, samples will be tested while submerged in saline at 37° C. Storage modulus and tan ∂ will be measured using frequency sweeps from 0.1-100 Hz at 0.2% dynamic strain. Stress-life (S-N) curves will be generated by cycling the materials at extreme, supra-physiological levels (n=3/stress level). Our expectation is that the properties of the materials will be close to or above the MTP joint cartilage, which is reported to have compressive modulus of 0.31-0.8 MPa and should not fail the fatigue testing under peak load of 4 MPa after 5 million continuous cycles. Each mechanical property data set will undergo statistical analysis using a 1-way ANOVA with Tukey's post-hoc test (p<0.05) using IgorPro. The results from this task will be used to identify the LCE compositions that fall within the range of the MTP joint's mechanical properties as stated above.

Task 2: Wear Study of LCEs

Following LCE synthesis, wear analysis of LCEs (n=5/group) will be performed to evaluate the effect of cushioning and impact absorption on volumetric wear rate, according to ASTM F732-17. This step is necessary since LCEs are a new class of material proposed for use in orthopedic implants. We will perform the initial wear testing using our pin-on-disc tribometer to screen for the LCE with lowest wear rate in a physiological environment (bovine blood serum at 37° C.). Per ASTM standard, LCE samples will be tested against polished cobalt-chromium-molybdenum (CoCrMo) alloy with an average contact stress of 3.54 MPa at 1 Hz for 5 million cycles (assuming 1 million cycles is 1 year of ambulation). The wear rate of LCEs before, during, and after testing will be determined (mm³/cycle), and the wear debris particulate collected during the LCE wear study will be analyzed for size, total quantity, and particle morphology using laser light scattering and scanning electron microscopy. The wear study results of LCEs will be compared to the ASTM standard of ultra-high molecular weight polyethylene (UHMWPE) against CoCrMo, which is commonly used in orthopedic implants. Comparisons will be made using a 2-way ANOVA with Tukey's post-hoc test (p<0.05) using IgorPro. The results from this task will be used to identify the LCE composition that has both similar properties to the MTP joint and has the lowest wear rate. It is noted that the volumetric wear rate of LCEs should be lower than UHMWPE which is 80 mm³/year.

Expected Outcomes

Successful completion of this aim will result in validating our claim to make LCEs that would mimic the MTP joint and significantly improve the stress distribution, shock absorption/impact loading damping, and more importantly, reduce wear rate observed in current MTP joint devices. Consequently, this approach can reduce the risk of complications caused by wear, increase longevity of the implants, and reduce the number of revisions in patients receiving MTP arthroplasty implants.

Preliminary Work and Results

Compression tests were performed on LCE samples (mono- and poly-domain) and compared to 1 cm biopsies of a cadaveric cartilaginous disc. Initial results showed that the LCE materials fall within the range of the natural cartilaginous disc. Testing cylindrical LCE samples (10 mm diameter×10 mm height) under cyclic compression of 3 MPa at body temperature at 1 Hz using DMA (Electroforce 3200, TA Instruments) has also been performed. Samples were press-fit into an HDPE fixture with 25% of the length exposed to simulate the MTP joint loading scenario. The stress vs. strain for various cycle counts were plotted. The cyclic loading result demonstrated accumulation of strain in first few cycles while hysteresis loop of the stress-strain plot indicated energy dissipation after 580,000th loading cycle (test is ongoing and will be stopped at 5 million cycle) making it very promising for MTP joint application.

Testing of 3D-printed LCE cylinders for spinal fusion cage and showed LCEs can withstand 1 million cycles at 2 Hz between 140-280 N load. These results further support use of LCEs for orthopedic application.

Alternative Strategies

Preliminary results showed that LCEs have the expected properties and match the properties of MTP joint. However, it is possible that a composite design may be incorporated, such as using a fibrous composite material to add an additional length scale anisotropy; this approach would consequently help achieve the desired outcome in matching the natural MTP joint. Additionally, LCEs can be made with a porous surface to permit additional areas for bone ingrowth, securing the implant over time and reducing the likelihood of expulsion.

Aim 2: Determine the Biomechanical Performance and Biocompatibility of LCE Devices

The goal of this aim is to validate the biomechanical performance and biocompatibility of the prototype LCE device against a competitor's device using relevant testing standards as required for regulatory clearance. Prototype LCE devices will undergo mechanical testing in comparison to the competitor's devices. Prototype LCE devices will be implanted into synthetic metatarsals to determine wear rate and to analyze any particulate wear debris. This aim will generate the necessary mechanical data for regulatory clearance and prove that the prototype LCE device has superior mechanical and wear properties compared to the competitor's device.

Working Example 2

LCE devices were molded into two sizes (8 mm diameter×8 mm height, 10 mm diameter×10 mm height) using the optimal LCE composition found in Aim 1. A series of mechanical tests were performed to determine the performance of the LCE device. A series of monotonic mechanical tests were performed to compare the LCE device to previous devices. First, unconfined compression was tested according to the methods of Korhonen 78 (n=5/device size), where the devices will be compressed incrementally to 10%, 20%, 30%, and 40% strain to determine the Young's modulus. The LCE device should have a modulus close to the previous devices modulus of 3.05±0.12 MPa. Second, confined compression will be tested 78 (n=5/device size), where the devices will be compressed incrementally to 5%, 10%, 15%, 20%, and 25% strain to determine the aggregate modulus. The LCE device should have an aggregate modulus equal to a conventional aggregate modulus of 6.7±1.0 MPa. Third, samples were tested in shear according to the study published on PVA used for Cartiva (n=5/device size), where the devices will be sheared to 100% shear at strain rates of 0.0125 s⁻¹ and 0.125 s⁻¹ to determine the shear tangent modulus. The LCE device should have a shear tangent modulus equal to or greater than the competitor's shear modulus of 0.16-0.36 MPa. Fourth, samples were tested in confined compression to determine creep behavior under a 4 MPa applied load (n=5/device size). The LCE device should have minimal dimensional change during the test, where the competitor's device showed a 5% change, and complete shape recovery should occur once applied load has been removed. Next, confined cyclic compression tests were run with maximum peak stresses of 24, 18, 12, 8, and 4 MPa to determine the stress-life curve of the LCE device. Run-out was determined when the LCE device can survive 5 million cycles (R=0.1, 5 Hz, n=2/stress level). The previous devices survived up to 5 million cycles at a stress level of 4 MPa, thus the LCE must meet run-out at this stress level or at a prior higher stress-level. Wear testing was run to simulate a worst-case scenario of 5 years of walking by applying 5 million cycles with a 4 MPa axial load. The LCE device was articulated against CoCrMo or bovine cartilage in a joint simulator with a cycle of ±650 to mimic the dorsiflexion experienced during walking. Mass loss and volumetric wear rate were determined for each sample (n=6/group). The mass loss and volumetric wear rate was determined and compared to the competitor's device. Wear particles were collected and analyzed following ASTM F1877 via scanning electron microscopy to determine particle shape, aspect ratio, and equivalent circle diameter. Initial biocompatibility assessment of the wear particles was conducted by a 3rd party laboratory under ISO 10993 standards for direct contact and elution cytotoxicity.

Expected Outcomes

Successful completion of this aim will result in verification of the biomechanical performance of the prototype LCE devices, including the capacity to survive the simulated cyclic loading and lower volumetric wear rate than the competitor's devices.

Preliminary Work and Results

Biocompatibility of the LCEs materials was confirmed by a third-party research institute, WuXi AppTec (St. Paul, MN), following ISO-10993 using an elution and direct-contact assay. The tested LCE networks were shown to have a cytocompatible response at both stages of two-step reaction.

Another measure of compatibility is in vivo subcutaneous implantation. The subcutaneous physiological response of solid and porous polydomain LCE materials was investigated by in vivo implantation in rats. Neither the solid nor porous samples showed any obvious inflammatory response or observable swelling. No adverse conditions as a result of implantation were observed and all rats were considered healthy throughout the trial period.

Additionally, the effect of simulated physiological conditions on the mechanical behavior of LCEs was investigated by comparing the material response before and after exposure (soaked in PBS at 37° C. for 12 days) during uniaxial tension and creep tests. In brief, the mechanical response for both tests are relatively stable and deviations from the pristine behavior were minimal.

The stable mechanical behavior in physiological conditions, noncytotoxic response, and inert behavior during subcutaneous implantation are all strong evidence towards validating the biological compatibility of LCEs for MTP application.

Coefficient of Friction

Coefficient of Friction (CoF) testing was conducted by an independent laboratory, Cambridge Polymer Group (Woburn, Massachusetts), under good laboratory practices (GLP) and with guidance from ISO 7148-2(2012), Method A. Testing utilized a rotational rheometer with pins-on-flat or pins-on-disc configuration in order to determine the CoF of LCE45 against cartilage and Cobalt-Chromium (CoCr). For this study, medical grade Ultra-High Molecular Weight Polyethylene (UHMWPE) and CoCr were used as control samples due to their wide use in orthopedic devices as a joint replacement material. LCE45 and UHMWPE flat cylindrical pins were tested against flat CoCr discs. Additionally, freshly harvested bovine cartilage flat cylindrical pins were tested against CoCr, LCE45, and UHMWPE flat/disc substrates.

Test conditions were in bovine synovial fluid at 37° C. with a 0.06 MPa normal stress applied for a duration of 5 minutes. All pin and substrate combinations were tested at two velocities: 10 mm/s and 100 mm/s and a sample size of n=3 per configuration. The average CoF for each material configuration at two speeds was tested (see Table 1).

TABLE 1
Average Coefficients of Friction

| | 10 mm/s | 100 mm/s |
|---|---|---|
| LCE on CoCr | 0.50 | 0.47 |
| UHMWPE on CoCr | 0.14 | 0.09 |
| Cartilage on CoCr | 0.30 | 0.01 |
| Cartilage on LCE | 0.09 | 0.02 |
| Cartilage on UHMWPE | 0.72 | 0.28 |

Overall, the CoF at the slower sliding velocity was higher for each material combination. At 10 mm/s sliding speed, cartilage had a lower CoF on the LCE substrate than cartilage on CoCr and UHMWPE substrates. At the faster 100 mm/s sliding speed, the CoF for the cartilage against CoCr and LCE substrates were comparable, and they were the lowest CoFs measured in this study ranging from 0.01 to 0.02. The highest CoF was measured for the cartilage against the UHMWPE substrate at 10 mm/s speed. The LCE on CoCr showed no rate dependency. Overall, the cartilage on LCE demonstrated low CoF and is the intended opposing surface for the LCE material.

Biological Testing of LCE45

Biocompatibility screening of LCE45 was performed following the International Standards Organization (ISO) standard for the biological evaluation of medical devices (ISO 10993-1) that has been adopted by both the FDA and the European Union (EU). Biocompatibility tests were performed under good laboratory practices (GLP) by Wuxi AppTec laboratories (St. Paul, MN, USA). The following tests determined whether the LCE material contains any toxic, leachable, or diffusible substances that can cause local or systemic response or can be absorbed into the circulatory system and cause systemic toxicity, mutagenesis, and tissue's immunological response. The characterization consists of a comprehensive battery of in vitro and in vivo tests, performed following standards outlined in ISO 10993-1:2018. For the LCE45, the battery of tests consisted of cytotoxicity, sensitization, intracutaneous irritation, intramuscular implantation with histopathology, materials-mediated pyrogenicity, acute systemic toxicity, subacute toxicity, genotoxicity 1 (in vitro mouse lymphoma), genotoxicity 2 (bacterial mutagenicity-Ames assay), and bacterial endotoxin (LAL).

LCE45 passed all the aforementioned tests. The results of the biocompatibility tests are presented in Table 2, followed by a brief description of each biocompatibility test and its results.

TABLE 2
Biocompatibility Test Results

| | Test | Results |
|---|---|---|
| LCE45 | Cytotoxicity | Passed |
| Testing | Intracutaneous Irritation | Passed |
| Summary | Sensitization | |
| | Guinea Pig Maximization | Passed |
| | Material-Mediated Pyrogenicity | Passed |
| | Acute Systemic Toxicity | Passed |
| | Subacute/Subchronic Toxicity | |
| | Intravenously | Passed |
| | Intraperitoneally | Passed |
| | Genotoxicity | |
| | In Vitro Mouse Lymphoma Assay | Passed |
| | Bacterial Mutagenicity - Ames Assay | Passed |
| | Implantation | |
| | Intramuscular Implantation with | Passed |
| | Histopathology in rabbits (13 weeks) | |
| | Bacterial Endotoxin (LAL) | Passed |

Cytotoxicity (Per ISO 10993-5)

Test Criteria

Cytotoxicity evaluates the effect of leachable and/or diffusible substances from a test article on the morphologic changes of mammalian cells, such as granulation, crenation, or rounding, and loss of viable cells from the monolayer by lysis or detachment by a method compliant with the requirements specified in ISO 10993-5:2009. According to current ISO guidelines, test articles scoring ‘0’, ‘1’, or ‘2’ will be considered ‘non-cytotoxic.’ Test articles scoring ‘3’ or ‘4’ will be regarded as ‘cytotoxic.’

Test Results

LCE45 scored ‘0’ at 24-, 48-, and 72-hour intervals and was non-cytotoxic. No abnormal events, such as pH change or debris, were noted. Conclusion: PASSED

Intracutaneous Reactivity (Irritation) (Per ISO 10993-10)

Test Criteria

The irritation test evaluates whether the test article can cause local irritation by applying the test article extracts directly to the animal and evaluating the irritation reactions. The injection site was observed immediately post-injection and at 24-, 48-, and 72-hour time intervals and included tissue reactions rated for gross evidence of erythema and edema. According to ISO 10993-10 test criteria, if the difference between the average scores for the extract of the test article and the vehicle control is less than or equal to 1.0, the test article is considered to have met the requirements of the test.

Test Results

None of the animals in this study showed abnormal clinical signs, and no dermal reactions were observed at the injected test and control sites on the rabbits at the 24-, 48-, and 72-hour observation periods. The averages for normal saline (NS) extracts were 0.0 for the test article and 0.0 for the control. The averages for the sesame oil (SO) were 1.2 for the test article and 1.0 for the control. The differences in the mean test and control scores of the extract dermal observations were 0.2, which is less than 1.0, indicating that the test article had met the ISO Intracutaneous Reactivity. Conclusion: PASSED

Guinea Pig Maximization (Sensitization) (Per ISO 10993-10)

Test Criteria

The sensitization test determines the sensitizing activity and the potential of a test article to cause a delayed hyper-sensitivity reaction by exposing the animals to the test article and evaluating the sensitization reactions. The test is used as a procedure for the screening of contact allergens in guinea pigs and extrapolating results for humans. Grades of ‘1’ or greater in the test group indicate sensitization.

Test Results

None of the animals challenged with the control and test article extracts were observed with a sensitization response greater than ‘0’. The test material's normal saline and sesame oil extract had a sensitization response of ‘0’ under valid test conditions. Under the conditions of this protocol, the test article did not elicit a sensitization response. Conclusion: PASSED

Materials Mediated Pyrogenicity (Per ISO 10993-11)

Test Criteria

Pyrogenicity determines if the test article extracts can cause a febrile response in animals.

The individual rabbit baseline temperature was subtracted from the maximum temperature recorded during 1- and 3-hour post-injection. If none of the rabbits had a temperature difference of less than 0.5° C., then the test article met the requirements for the absence of pyrogens. Failure is defined if any rabbit had an individual temperature rise greater than 0.5° C.

Test Results

The baseline temperatures for the rabbits were within 1° C. at the start of the test, and no animals had a baseline temperature above 39.8° C. or less than 38.5° C. During the 3-hour observation period, none of the rabbits administered with the test article extract had a temperature rise greater than 0.5° C. This response did not exceed the United States Pharmacopeia (USP) limit and met the requirements for this test. Therefore, these results indicated that the test article was non-pyrogenic. Conclusion: PASSED

Acute Systemic Toxicity (Per ISO 10993-11)

Test Criteria

Acute systemic toxicity evaluates the potential of a test article to cause adverse effects distant to the entry point by dosing the animals intravenously and/or intraperitoneally with test article extracts and monitoring for various signs of toxicity at different time intervals. According to ISO guidelines, the test is considered negative if none of the animals injected with the test article extract show a significantly greater biological reaction than the animals treated with the control vehicle. Death in two or more mice or other toxic signs such as convulsions, prostration, or body weight loss greater than 10% in three or more mice is interpreted as significant biological reactions. Observations for mortality and signs of pharmacological and/or toxicological effects were made immediately after injection and at 4-, 24-, 48-, and 72-hours post-injection. For this test, two extracts were used: normal saline and sesame oil.

Test Results

None of the animals in the study were observed with abnormal clinical signs indicative of toxicity during the 72-hour test period. All were alive at the end of the 72-hour test duration, and body weight changes were within acceptable parameters over the course of the study. These findings indicate that the test article has met the ISO Acute Systemic Injection Test requirements. Conclusion: PASSED

Subacute/Subchronic Toxicity (Per ISO 10993-11)

Test Criteria

The purpose of this study is to evaluate the systemic toxicity of leachable compounds from the test article. In order to evaluate the systemic toxicity of leachable compounds, animals were administered intravenously a normal saline extract and intraperitoneally a sesame oil extract. This test is intended for medical devices with a contact duration categorized as prolonged (24 hours to 30 days) or permanent (greater than 30 days). Each of the following parameters will be used as signs of toxicity; however, the final evaluation of the test article will involve consideration of all the data for patterns of toxicity. Repeated dosing of a test extract ensures longer-term (subchronic) exposure to material leachables than the typical ISO Acute Systemic Toxicity Test. Twenty-four rats (12 test/12 control) will be administered intravenously (Dose=10 mL/kg×14 intervals) a 0.9% normal saline extract of the test article or vehicle control 14 times over a 14-day test period. In addition, the same rats will also be administered intraperitoneally (Dose=5 mL/kg×5 doses) a sesame oil extract of the test article or vehicle control 5 times on days 1, 4, 7, 10, and 13. Animals will be observed once daily for signs of toxicity, death of more than one animal per group, mean body weight loss for each group, and clinical signs of toxicity in more than one animal per group. Hematology, coagulation, and clinical chemistry values for animal groups will be reviewed for a pattern of toxicity. Histopathology of tissues in the test and control groups will be evaluated for patterns of toxicity.

Test Results

All animals in the test and control groups survived for the duration of the 14-day study. No abnormal clinical signs were noted in any of the animals during daily observations. All animals gained weight during the 14-day study, and there was no mean body weight loss in any group over the course of the study. There were no statistically significant differences (all p-values were greater than 0.05) between the test and control rats' body weights at any recorded time period in the study. No abnormal findings were noted at necroscopy for the test animals. The pathology analysis concluded that no adverse findings could be attributed to the test article. Statistically significant differences were present in hematology, coagulation, clinical chemistry, and organ weight data; however, these findings were not considered to be indicative of a pattern of toxicity. The extracts of this test article are considered negative for signs of systemic toxicity due to leachable components under the conditions of this study. Conclusion: PASSED

Genotoxicity (Per ISO 10993-3)

In Vitro Mouse Lymphoma Assay

Test Criteria

Genotoxicity determines whether a test material can induce either point mutations or clastogenic (chromosomal breakage) events in cultured mouse lymphoma cells, which have the potential for cancer. The assay's objective is to determine a test article's ability to induce forward mutations of the thymidine kinase (TK) locus as assayed by colony growth of L5178Y mouse lymphoma cells in the presence of trifluorothymidine (TFT). Thymidine kinase (TK) is an enzyme that allows cells to salvage thymidine from the surrounding medium for DNA synthesis. If the thymidine analog TFT is included in the growth medium, the analog will be phosphorylated via the TK pathway and will cause cellular death by inhibiting DNA synthesis. Cells that are heterozygotes at the TK locus (TK+/−) may undergo a single-step forward mutation to the TK−/− genotype in which little or no TK activity remains. These mutants are as viable as the heterozygotes in a normal medium because DNA synthesis proceeds by de novo synthesis pathways that do not involve thymidine as an intermediate. TK−/− mutants cannot utilize toxic analogs of thymidine. Cells that may grow to form colonies in the presence of TFT are therefore assumed to have mutated, either spontaneously or as a result of exposure to the test article, at the TK+/−locus. Mouse lymphoma cells are used to determine whether a test material can induce either point mutations or clastogenic (chromosomal breakage) events in a cultured mammalian cell line.

Test Results

The test article is considered to be non-mutagenic and non-clastogenic for the test system in the saline test article extract in all treatment groups, dimethylsulfoxide (DMSO) test article extract in the 24-hour treatment group, DMSO test article extract 4-hour treatment group without metabolic activation (−S9) and the 3.17% and 1.00% dose levels of DMSO test article extract with metabolic activation (+S9). The controls for the assay were performed as required, qualifying both the assay and cell culture system as valid. The mutant frequencies and cloning efficiencies of preparations treated with saline test article extract and DMSO test article extract dilutions of 3.17% and 1.00% were within limits defined for a negative response. In the initial testing, the mutant frequencies for the saline test article extract in all treatment groups, DMSO test article extract in the 24-hour treatment group, and DMSO test article extract 4-hour treatment group without metabolic activation (S9), were not significantly different from those in the negative control groups. Additionally, in the confirmatory testing, the mutant frequencies of the DMSO test article extract 3.17% and 1.00% treated preparations in the 4-hour group with metabolic activation (+S9) were not significantly different from those in the negative control groups. Conclusion: PASSED

Bacterial Mutagenicity—Ames Assay

Test Criteria

This bacterial mutagenicity assessment (Ames Assay) evaluated the potential of the test article extracts to induce histidine (his) reversion in Salmonella typhimurium (his− to his+) or tryptophan (trp) reversion in Escherichia coli (trp− to trp+) caused by base changes or frameshift mutations in the genome of tester organisms. This plate incorporation assay was conducted with four strains of Salmonella typhimurium (TA97a, TA98, TA100, and TA1535) and one strain of Escherichia coli (WP2-uvrA-) in the presence and absence of S9 exogenous mammalian activation system. The test article was extracted in 0.9% sodium chloride (saline) and dimethylsulfoxide (DMSO). DMSO was chosen as the organic solvent for this assay after a pretest found the test article to be compatible. A range-finding study was not required for this assay, as the neat extract of a medical device represents a worst-case scenario.

Test Results

The test article was assayed as undiluted extracts. Spontaneous reversion rates of the negative control treated plates were within acceptable laboratory historical ranges, and all positive controls showed suitably increased reversion rates, indicating a valid assay. Therefore, this assay was capable of correctly identifying potential mutagens and non-mutagens. In this assay, the test article did not induce substantial increases in reversion rates of the type that are associated with mutagenesis. Furthermore, no substantial test article toxicity was noted that may have interfered with the ability of the test system to detect mutagens. As none of the tester strains showed an increase in reversion rates when treated with the test article, the test article is determined not to have caused an increase in point mutations, exchanges, or deletions. Based on the criteria and conditions of the study protocol, the test article was non-mutagenic. Conclusion: PASSED

Intramuscular Implantation with Histopathology (13 weeks) (Per ISO 10993-6)

Test Criteria

The implantation test assesses the local pathological effects on living tissue induced by the test article when surgically implanted for an extended duration of time by using a microscopic examination of the exposed tissue. The final analysis of this test was based on the histological comparison of the test article to the comparison control material in 3 rabbits.

Test Results

All 3 rabbits survived to the study endpoint. Based on the histopathologic evaluations, the test article was considered a non-irritant. In macroscopic observations, all the explant observations were normal. In microscopic observations, the average of the test article site scores was 6.0, and the average of the control material, which was USP (United States Pharmacopeia) high-density polyethylene RS control plastic, was 5.8, resulting in an irritant ranking score of 0.2 for the test article. An irritant ranking score of 0.2 indicated the test article was a non-irritant compared to the USP high-density polyethylene reference standard. Conclusion: PASSED

Bacterial endotoxin (LAL) (Per ISO 10993-11)

Test Criteria

In biomedical manufacturing, products must undergo Bacterial Endotoxins Testing (BET) to meet appropriate safety standards before being released to the market. The Limulus Amebocyte Lysate (LAL) assay was first developed for BET to determine the presence/absence of endotoxin in a sample and to reveal how much endotoxin is present quantitatively. Pyrogens are fever-producing materials that most often originate from Gram-negative bacterial cell walls but can also originate as leachates from some chemicals and materials. Pyrogens from bacterial cell walls (the most commonly encountered type of pyrogen) are referred to as bacterial endotoxins and are readily detected by LAL. For this LAL, kinetic chromogenic LAL assay was utilized to detect the Gram-negative bacterial endotoxin qualitatively. The presence of the endotoxin causes a color change, and the solution becomes yellow.

Test Results

The LAL result showed less than 3.552 EU/Device (EU=Unit of measurement for endotoxin activity) endotoxin level at the dilution ratio of 1:20 for three LCE45 samples tested. This result is well below the United States Pharmacopeia (USP) requirement level of less than 20 EU/Device for medical devices that directly or indirectly contact the cardiovascular and lymphatic systems. Conclusion: PASSED

Biological Testing of MTP Device (LCE45 and Titanium)

Biocompatibility screening of LCE45 and Titanium was performed following the International Standards Organization (ISO) standard for the biological evaluation of medical devices (ISO 10993-1) that has been adopted by both the FDA and the European Union (EU). Biocompatibility tests were performed under good laboratory practices (GLP) by Wuxi AppTec laboratories (St. Paul, MN, USA). The following tests determined whether the LCE material contains any toxic, leachable, or diffusible substances that can cause local or systemic response or can be absorbed into the circulatory system and cause systemic toxicity, mutagenesis, and tissue's immunological response. The characterization consists of a comprehensive battery of in vitro and in vivo tests, performed following standards outlined in ISO 10993-1:2018. For the LCE45 and titanium, the battery of tests consisted of cytotoxicity, sensitization, intracutaneous irritation, intramuscular implantation with histopathology, acute systemic toxicity, bacterial endotoxin (LAL), and EO residual testing.

LCE45 and titanium stem passed all the aforementioned tests. The results of the biocompatibility tests are presented in Table 3.

TABLE 3
Biocompatibility of LCE45 and Titanium

| | Test | Results |
|---|---|---|
| LCE45 | Cytotoxicity | Passed |
| and | Intracutaneous Irritation | Passed |
| Titanium | Sensitization | |
| Testing | Guinea Pig Maximization | Passed |
| Summary | Acute Systemic Toxicity | Passed |
| | Implantation | |
| | Intramuscular Implantation with | Passed |
| | Histopathology in rabbits (4 and 26 | |
| | weeks) | |
| | EO residual Testing | Passed |
| | Bacterial Endotoxin (LAL) | Passed |

Cytotoxicity (Per ISO 10993-5)

Table 4 below shows the summary of all the biocompatibility testing for LCE45 for the FDA Material Master File submission and LCE45 and Titanium for the FDA 510(k) submission.

TABLE 4
Biocompatibility results of LCE45
and MTP Device (LCE45 + Titanium)

| Battery of Biocompatibility | Tier 1: LCE45 for | Tier 2: Final Device |
|---|---|---|
| Tests for LCE45 and Final | FDA Material | (LCE45 + Titanium) |
| Device | Master File | for FDA 510(k) |
| Cytotoxicity | Passed | Passed |
| Sensitization | Passed | Passed |
| Intracutaneous Irritation | Passed | Passed |
| Intramuscular implantation | Not Required | Passed |
| with Histopathology 4 weeks | | |
| Intramuscular implantation | Passed | Not Required |
| with Histopathology 13 weeks | | |
| Intramuscular implantation | Not Required | Passed |
| with Histopathology 26 weeks | | |
| Materials mediated | Passed | Not Required |
| pyrogenicity | | |
| Acute systemic toxicity | Passed | Passed |
| Subacute toxicity | Passed | Not Required |
| Genotoxicity 1 (in vitro | Passed | Not Required |
| mouse lymphoma) | | |
| Genotoxicity 2 (bacterial | Passed | Not Required |
| mutagenicity-Ames assay) | | |
| Bacterial endotoxin (LAL) | Passed | Passed |
| Exhaustive extraction/ | Completed/Passed | Not Required |
| chemical characterization | | |
| Toxicological risk assessment | Completed/Passed | Not Required |
| Chronic Toxicity | Not Required | Not Required |
| EO Residual Testing | Not Required | Passed |

Chemical Characterization (Per ISO 10993-18)

Chemical characterization of materials is recommended by ISO 10993-18 for biomaterials intended for prolonged use as implants. LCE45 was characterized by evaluating extracted chemicals obtained using three solvents—polar (purified water), mid-polar (ethanol), and non-polar (hexane). Qualitative and quantitative analyses utilizing gas and liquid chromatography (GC, UHPLC), mass spectroscopy (MS), and Fourier transform infrared spectroscopy (FTTR) were used to evaluate volatile, semi-volatile, and non-volatile organic compounds that might leach from the sample extracted in different solvents. For this test, NAMSA (Northwood, OH) was utilized for the extraction, analyses, and toxicological risk assessment.

Toxicological Risk Assessment (Per ISO 10993-17)

The toxicological risk assessment was performed on LCE45 and is based on chemical characterization testing. The risk assessment evaluates the type and levels of the chemicals based on ISO 10993-17 and ISO 21726. The risk assessment focuses on the extraction results and the level of safety for each chemical present. It also uses the literature to research extractables identified in the chemical characterization and calculates margins of safety for these chemicals whenever possible. This assessment provides information about the toxicity and pharmacokinetics of each compound, identifies potentially toxic chemicals, and estimates the limits or appropriate toxicological thresholds of the chemicals for prolonged exposure to the patient in a worst-case scenario (i.e., largest device and maximum device number). Based on the chemical characterization data, the toxicological risk assessment indicated that the risk of systemic toxicity, carcinogenicity, and reproductive/developmental toxicity associated with patient exposure to extractables from the LCE45 is negligible. Additionally, biological testing highlighted in section 10 indicates that the risk of genotoxicity is negligible.

Risk Assessment (Per ISO 10993-1)

The LCE45 material is intended to restore the native function and biomechanics of the MTP joint. It has proven biocompatibility per ISO 10993 for permanent (>30 days) implantable materials in contact with tissue or bone. The toxicological risk assessment evaluated the LCE material with an equivalent exposure of two devices in the body. All testing was done on sterilized LCE45 material, and the toxicological risk assessment indicated that the risk of systemic toxicity and reproductive/developmental toxicity from patient exposure to extractables from the LCE is negligible.

A goal is to create a hemi-cap device that restores the native function and biomechanics of the MTP joint. Current devices remove bone and cartilage from the distal end of the metatarsal. The devices are implanted at the end of the metatarsal to restore the shape of the bone; however, the function of the removed articular cartilage is not fully addressed. Cobalt chromium and UHMWPE materials have shown the ability to resist fatigue and wear, but fundamentally do not replicate the soft and shock-absorbing properties of soft tissues. LCE was chosen and designed to create an implant that can mimic the biomechanics of the joint and minimize stress concentrations and impacts to the joint that may ultimately lead to device failure and the need for revision surgery or fusion.

The raw materials for each batch of LCE are traceable to verify the chemistry and quality is maintained. All raw materials are measured using a calibrated high-precision scale and recorded to ensure batch to batch consistency. Calibrated ovens with specified temperatures are used in the manufacturing process. The curing process is time dependent, and the minimum time is monitored to ensure the reaction completes. The LCE45 manufacturing process is validated with gel fraction measurements, dynamic mechanical analysis, and tensile testing to confirm the proper structure, properties, and processing. The quality controls outlined in the manufacturing process and the post-process testing conducted, help to mitigate potential inconsistencies in the LCE batches.

LCE can be cast or over-molded into a semi or finished form. The material may be cut or trimmed into a finished material/device as necessary. The final device's surface finish will depend on the material used for casting/molding. There are no secondary modifications to the surface. Molds made from materials such as glass or mirror-finish metal with a surface roughness range of 2-12 Ra have been used for molding final material/device products. No other mold-release substances or materials were used in the manufacturing process.

Measurements are taken of the material or device in its final form. The final material or device is then packaged for sterilization. As part of qualification activities, LCE45 underwent three sterilization methods as part of characterization: steam (autoclave), ethylene oxide, and irradiation. Dynamic mechanical analysis and FTIR-ATR showed the sterilization process did not affect the material chemically or mechanically.

MTP-LCE Devices

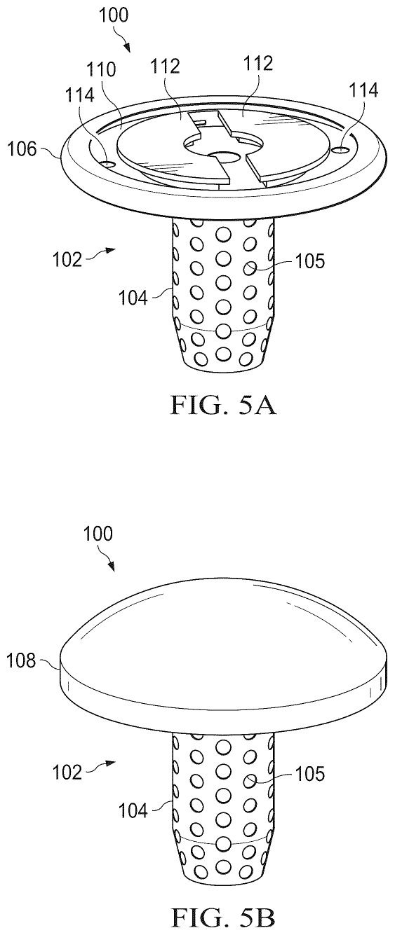

FIGS. 5A and 5B illustrate an MTP device 100 according to aspects of the disclosure. MTP device 100 may be used to treat osteoarthritis of an MTP joint. MTP device 100 includes a body 102 with a stem 104 and a head 106. Stem 104 is generally cylindrical, hollow, and includes a plurality of perforations 105 that promote osseointegration. In some aspects, body 102 is made of titanium. In some aspects, the titanium may be treated with a titanium plasma spray that creates a rough surface that promotes osseointegration. Stem 104 tapers at its distal end. Head 106 is affixed at a proximal end of stem 104 and includes features that promote bonding with an LCE cap 108. In a typical aspect, LCE cap 108 is molded onto head 106. Features include, for example, a cavity 110 that receives LCE when it is molded onto head 106. FIG. 5A illustrates two islands 112 positioned above cavity 110 that further help bond the LCE to head 106. Once the LCE has cured, a portion of the cured LCE is located underneath islands 112 making it difficult for the LCE to separate from head 106. In other aspects, the number and shape of islands 112 may be changed. Cavity 110 includes a plurality of perforations 114 that allow LCE to flow through. Once the LCE cures, the plurality of perforations 114 further secure LCE cap 108 to head 106. In some aspects, the plurality of perforations 114 may be countersunk on an underside of head 106 so that the cured LCE cannot pull out of the plurality of perforations 114.

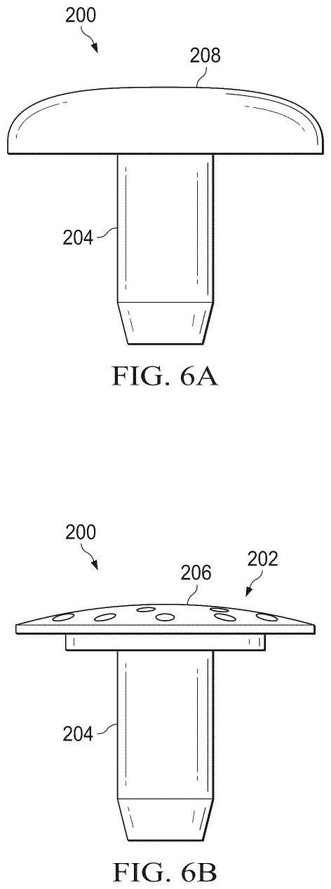

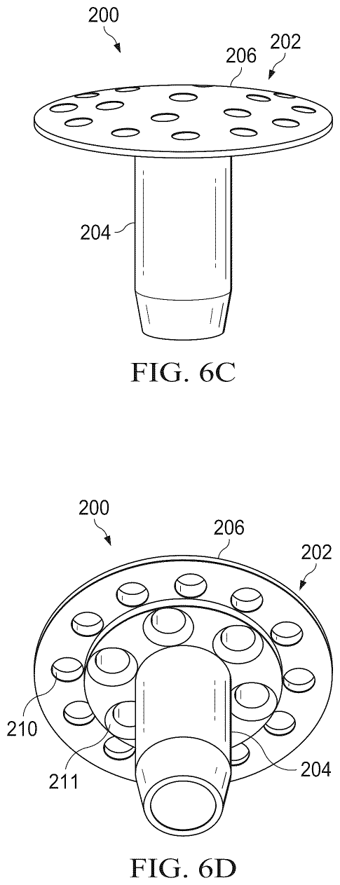

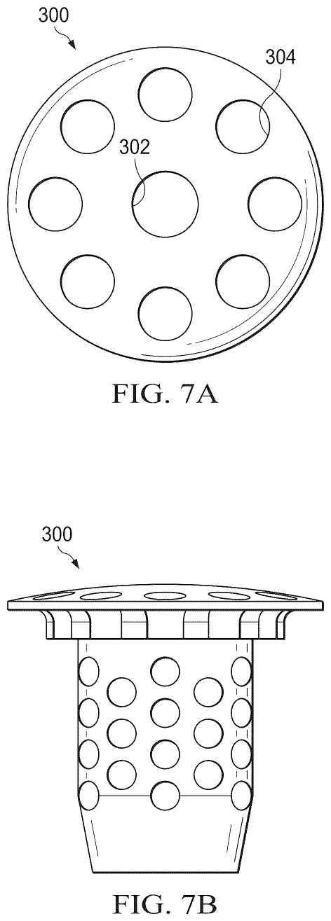

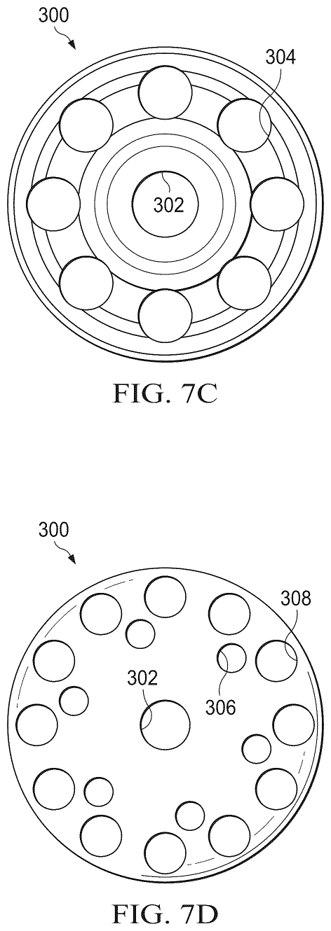

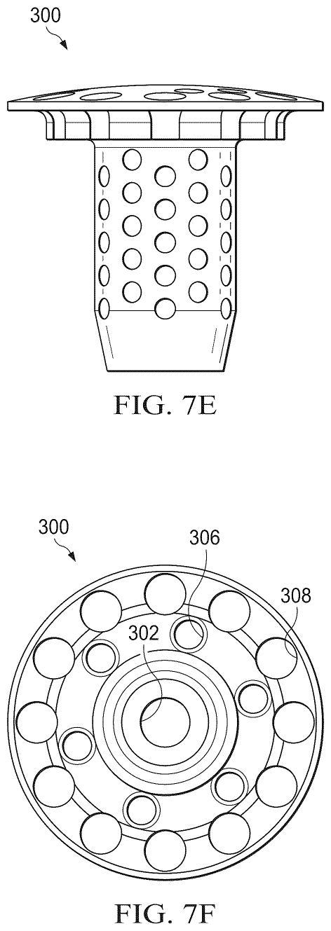

FIGS. 6A-6D illustrate multiple views of an MTP device 200 according to aspects of the disclosure. MTP device 200 may be used to treat osteoarthritis of an MTP joint. MTP device 200 includes a body 202 with a stem 204 and a head 206. Stem 204 is generally cylindrical and hollow. Stem 204 is illustrated without perforations, but in other aspects may include perforations similar to stem 104. Stem 204 tapers at a distal end. MTP device 200 includes an LCE cap 208 that is molded onto a proximal end of head 206. Head 206 includes a plurality of perforations 210 around a periphery of head 206 and a plurality of perforations 211 that are radially inside the plurality of perforations 210. As shown in FIG. 6D, the plurality of perforations 211 are countersunk on an underside of head 206 to promote bonding between the LCE and head 206. In other aspects, the plurality of perforations 210 may be countersunk. As best seen in FIGS. 6B and 6D, an inner portion of head 206 has a greater thickness than an outer portion of head 206.

In various aspects, MTP devices 100/200 may be modified. The variations include different lengths for the stems, different diameters for the heads, and different configurations of the plurality of perforations (e.g., different number, sizes, positions).

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded._{#}

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein._{#}

What is claimed is:

1. A device for a metatarsophalangeal (MTP) joint, the device comprising:
a stem;
a head located at a proximal end of the stem and comprising a plurality of perforations formed through the head; and
a liquid-crystalline elastomer cap formed over the head.

2. The device of claim 1, wherein the head includes an inner portion having a greater thickness than an outer portion.

3. The device of claim 1, further comprising a plurality of perforations formed through the stem.

4. The device of claim 1, wherein the stem comprises a threaded surface.

5. The device of claim 1, wherein the stem comprises a radially extending prong.

6. The device of claim 3, wherein at least one perforation of the plurality of perforations is countersunk.

7. The device of claim 3, wherein at least one perforation of the plurality of perforations is inline with a hollow of the stem.

8. The device of claim 3, wherein the plurality of perforations includes perforations of at least two different diameters.

9. The device of claim 1, wherein the head includes a countersunk bore that is axially aligned with a bore of the stem.

10. The device of claim 1, wherein the head comprises an island around which LCE of the LCE cap may cure to secure the LCE cap to the head.

11. The device of claim 1, wherein the LCE cap is formed over a lip of the head.

12. The device of claim 1, wherein the stem is tapered at a distal end.

13. A method of making a device for an MTP joint, the method comprising:
forming a body comprising a stem and a head;
applying a LCE to the head,
wherein the head comprises at least one feature to promote bonding of the LCE to the head.

14. The method of claim 13, wherein the at least one feature comprises a perforation formed through the head.

15. The method of claim 13, wherein the at least one feature comprises a first plurality of perforations and a second plurality of perforations, the first plurality of perforations positioned at a first distance from a central axis of the stem and the second plurality of perforations positioned at a second distance from the central axis of the stem that is different than the first distance.

16. The method of claim 15, wherein each perforation of the first plurality of perforations has a first diameter and each perforation of the second plurality of perforations has a second diameter that is different than the first diameter.

17. The method of claim 14, wherein the perforation is countersunk.

18. The method of claim 13, wherein the stem is hollow and includes a perforation formed through a wall of the stem.

19. The method of claim 13, wherein the head includes an island under which a portion of the LCE may flow.

20. A method of treating osteoarthritis of an MTP joint, the method comprising inserting the device of claim 1 into a bone forming the MTP joint.
